(19) United States
(12) Patent Application Publication
(10) Pub. No.: US 2020/0000699 A1
(43) Pub. Date: Jan. 2, 2020

(54) BIOACTIVE COMPOSITIONS AND METHODS FOR TREATING SKIN

(71) Applicant: ZO SKIN HEALTH, INC., Irvine, CA (US)

(72) Inventors: Frederick W. WOODIN, JR., Wheatland, WY (US); Maria Concepcion STAHL, Redondo Beach, CA (US)

(21) Appl. No.: 16/454,699

(22) Filed: Jun. 27, 2019

Related U.S. Application Data
(63) Continuation of application No. 16/379,371, filed on Apr. 9, 2019.
(60) Provisional application No. 62/691,345, filed on Jun. 28, 2018.

(51) Int. Cl. A61K 8/64; A61K 8/24; A61K 8/58; A61K 8/73; A61K 8/9789; A61Q 19/00; A61K 8/893; A61K 8/20; A61K 8/23; A61K 8/34; A61K 8/49

(52) U.S. Cl. CPC A61K 8/64; A61K 8/24; A61K 8/585; A61K 8/73; A61K 8/9789; A61K 8/498; A61K 8/893; A61K 8/20; A61K 8/23; A61K 8/345; A61Q 19/007

(57) ABSTRACT

Described are topical compositions optionally containing glycosaminoglycan stimulators, anti-aging agents, cosmetic peptides, and skin-conditioning agents, as well as a kit and methods for treating skin including administering described topical compositions.

### CROSS-REFERENCE TO RELATED APPLICATIONS

This is a continuation application and claims priority to and the benefit of U.S. patent application Ser. No. 16/379,371, filed Apr. 9, 2019, which claims benefit to Provisional Patent Application Ser. No. 62/691,345 filed Jun. 28, 2018, which are incorporated herein by reference in their entirety.

### FIELD

The present subject matter relates to bioactive topical compositions and methods of treating the skin of a mammal by administering the same.

### BACKGROUND

The skin is the body's largest organ and is comprised of three structures: the epidermis, the dermis and the hypodermis. The epidermis is the outermost structure and contains various layers, of which the stratum corneum (SC) is the outermost layer, and serves as the rate-controlling barrier to limit the entrance of foreign substances as well as to facilitate the excretion of endogenous substances. The epidermal barrier can be altered by increasing age, environmental factors, or other conditions, which can result in a change in the appearance of the skin and function of the SC and epidermal barrier. The dermis is also susceptible to structural alterations with age and environmental exposure resulting in a loss of structural integrity that leads to the appearance of coarse wrinkles, fine lines and uneven skin texture. These changes can result in the onset of accelerated signs of skin aging which typically begin to appear in a patient's 20s and 30s and present as dull skin, fine lines, coarse wrinkles, loss of moisture and dry, rough skin texture.

Therefore, there is a need to identify compositions and methods that can be used to improve the signs of aging both immediately and for sustained periods, preferably without irritation.

### SUMMARY

The present subject matter relates to topical compositions and methods for treating skin including administering a topical composition.

More specifically, provided is a topical composition comprising or consisting of a combination of at least four bioactive elements such as, e.g., disodium acetyl glucosamine phosphate, lactobacillus/panax ginseng root extract ferment filtrate, dipeptide diaminobutyroyl benzylamide diacetate, hydrolyzed sericin, angelica polymorpha sinensis root extract, and beta-glucan, among others. As evident, more than one of the bioactive elements may be an enzymatically or plant derived growth factor.

The topical compositions may optionally comprise or consist of one or more additional agents. For example, the topical composition may optionally comprise or consist of a carrier, which may be a solvent, a chelating agent, a humectant, a surfactant, a viscosity adjusting agent, a glycosaminoglycan (GAG) stimulator, a buffering agent, a thickener, an emollient, an emulsifier, a skin conditioning agent, an anti-inflammatory agent, an anti-aging agent, an anti-wrinkle agent, a DNA repair agent, an antioxidant, a pH adjuster, a fragrance and/or a preservative. In addition, the topical composition may also comprise or consist of one or more biocompatible carrier, excipient, filler and/or diluent.

The presently provided compositions may be free from a retinoid such as retinol.

Also provided are methods of improving skin firmness and/or elasticity, methods for improving skin radiance and/or luminosity, methods for reducing fine lines and/or wrinkles, methods for reducing coarse wrinkles, methods for improving hydration, and methods for modifying skin density. Each of the methods may include administering a therapeutically effective amount of a composition described herein to a subject.

Also provided are skin kits. The skin kits may comprise or consist of the topical composition provided herein and one or more additional composition. The one or more additional composition may be a skin cleanser, an oral composition for treating skin, a sunscreen such as, e.g., an organic sunscreen, a moisturizing composition, an exfoliating composition such as, e.g., an exfoliating polish, a barrier protecting composition and/or an additional topical composition for treating, preventing or ameliorating a skin condition. The kits may also include a device for concomitant therapy. Additionally the kits may include one or more topical or oral homeopathic composition. The kits may further include instructions for use and packaging.

### DETAILED DESCRIPTION

### Definitions

The term “about” as used herein refers to a quantity, level, value, dimension, size, or amount that varies to some extent based on the context in which it is used. For example, such variation can be by as much as 5%. At the least, each numerical parameter can be construed in light of the number of reported significant digits and by applying ordinary rounding techniques.

As used herein, the terms “administer,” “administering,” and “administration,” refer to any method which, in sound medical practice, delivers the composition to a subject in such a manner as to provide a therapeutic effect.

For the purposes of the presently described subject matter, the present topical biocompatible compositions can be topically administered. For example, administration can include, but is not limited to, direct topical administration. For example, a viscous formulation, including for example a cream, gel, ointment, or salve formulation, or a liquid formulation, for example, a solution, can be administered directly to a desired skin surface. Administration can also be accomplished via a compress or a wet dressing. For example, a liquid, e.g., solution, emulsion, suspension, etc., formulation of the presently described topical composition can be applied to a skin surface via a compress or wet dressing.

As used herein, the phrases “anti-inflammatory agent” and “inflammatory management agent” refer to a compound or composition capable of preventing, reducing or ameliorating inflammation. Non-limiting examples of inflammatory management agents include, e.g., lactose and milk protein or a combination thereof. Anti-inflammatory agent may also refer to, for example, a chemical compound that acts to reduce one or more indications of inflammation, such indications including, but not limited to, swelling, redness, tenderness, and pain. Such compounds can include but are not limited to Beta Glucan, lactose and milk protein, Avena Sativa (Oat) Kernel Meal, Avena Sativa (Oat) Kernel Protein USP, Avena sativa (Oat) Kernel Flour, whey protein concentrates, and combinations thereof.

As used herein, the term “anti-aging agent” means a compound or composition that inhibits or reduces signs of aging, such as wrinkles, fine lines, and other manifestations of photodamage. Examples of anti-aging agents include, but are not limited to, the following compounds and compositions that include the same: natural red ginseng extract; lactobacillus/panax ginseng root extract ferment filtrate; BIO-FRGE®; angelica polymorpha sinensis root extract; DC Provesselin; flavonoids such as quercetin, hesperidin, quercitrin, rutin, tangeritin, and epicatechin; CoQ10; inorganic sunscreens such as titanium dioxide and zinc oxide; organic sunscreens such as octyl-methyl cinnamate and derivatives thereof. Anti-aging agents may comprise antioxidants. Non-limiting examples of antioxidants include one or more of Leontopodium Alpinum Meristem Cell Culture, Marrubium Vulgare Meristem Cell Culture (e.g., DISTINCTIVE® Phytostem Edelweiss), Tocopheryl Acetate, Ascorbic Acid and/or Retinyl Palmitate, soluble soy protein, Idebenone, coenzyme Q10, Lycopene, Epigallocatechin 3-gallate (EGCG), green tea polyphenols (GTP), Silymarin, COFFEEBERRY®, buddleja davidii meristem stem cell, Grape seed extract, Pomegranate extracts, Genistein, pycnogenol, niacinamide, methionine, glutathione, tocotrienol, dimethylglycine, betaine, butylated hydroxyanisole, butylated hydroxytoluene, turmerin, vitamin E, ascorbyl palmitate, deteroxime mesylate, butylated hydroxyanisole, butylated hydroxytoluene, propyl gallate, sodium or potassium metabisulfite, sodium or potassium sulfite, alpha tocopherol or derivatives thereof, sodium ascorbate, disodium edetate, BHA (butylated hydroxyanisole), flavonoids, a pharmaceutically acceptable salt or ester of the mentioned compounds, and mixtures thereof. Flavonoids include, for example, quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin, epigallocatechin gallate, flavonol, epicatechin, hesperetin, chrysin, diosmin, hesperidin, luteolin, and rutin. Exfoliants may include alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrolactic acid, alpha-hydroxyisovaleric acid, ethyl pyruvate, galacturonic acid, glucoheptonic acid, glucopheptono-1,4-lactone, gluconic acid, gluconolactone, glucuronic acid, glucuronolactone, glycolic acid, isopropyl pyruvate, methyl pyruvate, mucic acid, pyruvic acid, saccharic acid, saccharic acid 1,4-lactone, tartaric acid, and tartronic acid; beta hydroxy acids such as beta-hydroxybutyric acid, beta-phenyllactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, pueraria, rice, safflower, and mixtures thereof.

The term “biocompatible” or “physiologically compatible,” as used herein, refers to the ability to be in contact with a living system without producing a significant adverse effect, for example, by not being toxic, injurious, or physiologically reactive.

As used herein, a “cosmetic peptide agent” or “cosmetic peptide” means any peptide, or agent including a peptide, that is known to block neuromuscular transmission. Non-limiting examples of “cosmetic peptides” include, for example: argireline analogues obtained from protein SNAP 25 (see EP 1180524), pentapeptide leuphasyl developed by LIPOTEC COMPANY (Spain), which mimics enkephalin action decreasing neuronal excitation by inhibiting Ca²⁺-influx throughout the membrane and decreasing Ca²⁺-dependant transmitter release; Inyline, which is an acetyl hexapeptide developed by LIPOTEC COMPANY (Spain); vialox (Pentapeptide-3V by PENTAPHARM COMPANY, Switzerland), which is a pentapeptide fragment of the neurotoxin waglerin-1 from the venom of the Temple Viper; Dipeptide Diaminobutyroyl Benzylamide Diacetate and compositions including the same, such as, SYN-AKE, which is a reversible antagonist of the muscle-type nicotinic acetylcholine receptor (mnAChR). SYN-AKE is a tripeptide that acts in a manner similar to waglerin-1, which prevents acetylcholine binding with the receptor and the channel activation.

“DNA repair agent” as used herein refers to an agent, e.g., a chemical, that includes one or more amino acids that help repair visible UV-induced DNA damage. A DNA repair agent according to the present subject matter may include, e.g., a combination of Acetyl Tyrosine, Proline, with Hydrolyzed Vegetable Protein, Adenosine Triphosphate, commercially available as UNIREPAIR® T-43, or the combination of panthenyl triacetate and ethyl linoleate dissolved in oleyl alcohol, commercially available as UNIPROTECT PT-3® (both from Induchem).

As used herein, the phrases an “effective amount” or a “therapeutically effective amount” of a composition and/or active agent or ingredient, which are synonymous herein, refer to an amount of the active agent sufficient enough to have a therapeutic effect upon administration. A therapeutically effective amount of the active agent may, will, or is expected to cause a relief of symptoms. Effective amounts of the active agent will vary with the particular condition or conditions being treated, the severity of the condition, the duration of the treatment, the specific components of the composition being used, and like factors. For example, the presently described compositions can be topically applied in an amount sufficient to cover an affected area. The presently described compositions can be topically applied in an amount sufficient to cover an affected area plus a margin of healthy skin or tissue surrounding the affected area, for example, a margin of about 0.5 inches.

As used herein the term “emollient” refers to any product applied to the skin which softens and/or soothes irritation of the skin, including, for example, ointments, liniments, lotions, creams, moisturizers, oils, skin softeners, soaps, shampoo, cleansers, sunscreens, cosmetics and the like. Non-limiting examples of emollients suitable for the biocompatible topical compositions herein include, for example, one or more of Aleurites Moluccana Seed Oil, Carthamus Tinctorius (Safflower) Seed Oil, Isohexadecane, Methylheptyl Isostearate, Neopentyl Glycol Diethylhexanoate, Neopentyl Glycol Diisostearate and/or C12-15 Alkyl Benzoate, Hydrogenated Polyisobutene, C-12-C-15 alcohol benzoates, isopropyl myristate, mineral oils, lanolin and lanolin derivatives, and triglycerides such as coconut oil, cetostearyl alcohol, cetyl alcohol, isopropyl palmitate, caprylic/capric triglyceride, PPG-2 myristyl ether propionate, dimethicone, methicone, petrolatum, lanolin, and mineral oil.

As used herein “emulsifier” refers to a compound or substance that acts as a stabilizer for emulsions preventing the liquids from separating. Non-limiting examples of emulsifiers suitable for the present biocompatible topical compositions include, for example one or more of PEG-100 Stearate, PEG-15/Lauryl Dimethicone Crosspolymer, Polyglyceryl-4 Isostearate and/or Cetyl PEG/PPG 10/1 Dimethicone and/or Hexyl Laurate, polyoxyethylene fatty ethers derived from stearyl alcohols, Isopropyl Isostearate, Cetyl Alcohol, polyethylene glycol stearate, glycol stearate, glyceryl stearate, cetearyl alcohol, ceteareth 20, methylcellulose, cetomacrogol 1000, and lecithin.

As provided herein, a “glycosaminoglycan (GAG) stimulator” or “GAG stimulator” refers to any substance that stimulates the production of glycosaminoglycans. For example, the GAG stimulator may be one or more of, for example, disodium acetyl glucosamine phosphate (DAGP), hydrolyzed sericin, sericin, hyaluronic acid and derivatives or salts thereof, SUBLISKIN® (Sederma, INCI (International Nomenclature of Cosmetic Ingredients): Sinorhizobium Meliloti Ferment Filtrate, Cetyl Hydroxyethylcellulose, Lecithin), HYALUFIX® (BASF, INCI: Water, Butylene Glycol, Alpinia galanga leaf extract, Xanthan Gum, Caprylic/Capric Triglyceride), STIMULHYAL® (Soliance, INCI: Calcium ketogluconate), SYN-GLYCAN® (DSM, INCI: Tetradecyl Aminobutyroylvalyl-aminobutyric Urea Trifluoroacetate, Glycerin, Magnesium chloride), KALPARIANE® (Biotech Marine), DC Upregulex (Distinctive Cosmetic Ingredients, INCI: Water, Butylene Glycol, Phospholipids, Hydrolyzed Sericin), DC Upregulex-Buddeja Complex (Distinctive Cosmetic Ingredients, INCI: water, glycerin, hydrolyzed sericin, phospholipids, butylene glycol, buddleja davidii meristem cell culture, xanthan gum, phenoxyethanol, caprylyl glycol, potassium sorbate and hexylene glycol), glucosamine, N-acetyl glucosamine, Arctium lappa fruit extract, Eriobotrya japonica extract, Genkwanin, N-Methyl-L-serine, (−)-alpha-bisabolol or synthetic alpha-bisabolol such as, e.g., DRAGOSANTOL® and DRAGOSANTOL 100® from Symrise, oat glucan, Echinacea purpurea extract and soy protein hydrolysate, Sinorhizobium Meliloti Ferment Filtrate, Calcium ketogluconate, Alpinia galanga leaf extract and tetradecyl aminobutyroylvalylaminobutyric urea trifluoroacetate.

The term “humectant” refers to a substance capable of reducing the loss of moisture. Non-limiting examples of humectants include, for example, glycerin, sodium hyaluronate, glycerol, glyceryl triacetate, a lanolin product, such as PPG-12-PEG 50, polyhydric alcohols, water soluble alkoxylated nonionic polymers, and mixtures thereof. Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof. Commercially available humectants herein include: glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate with tradenames ACTIMOIST available from Active Organics, AVIAN SODIUM HYALURONATE series available from Intergen, HYALURONIC ACID Na available from Ichimaru Pharcos; sodium adenosine phosphate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Dow Chemical.

As used herein, the phrase “exfoliating agent” refers to any mechanical, chemical or naturally derived agent that is applied to the skin to accelerate the removal of dead cells from the skin surface to relieve dry skin, assist removal of papules or pustules, and/or reduce wrinkles. The exfoliating agent may include at least one plant-derived cysteine protease enzyme.

As used herein. the terms “pH adjuster” and “neutralizing agent” refer to any composition, compound or agent suitable for adjusting the pH of the presently described topical compositions without negatively affecting any property thereof. Suitable pH adjusters can include any acid or base. Suitable pH adjusters can include, but are not limited to, one or more of sodium hydroxide, aminomethyl propanol, hydrochloric acid, sulfuric acid, citric acid, acetic acid, formic acid, phosphoric acid, tartaric acid, and triethanolamine.

“Solvent” or “carrier” refers to inorganic and/or organic molecules and compounds capable of at least partially dissolving another substance (i.e., the solute). Solvents may be liquids at room temperature. Non-limiting examples of solvents include, for example, one or more of water, deionized water, hydrocarbon solvents (e.g., n-pentane, n-hexane, n-heptane, n-octane, paraffin, cyclohexane, methylcyclohexane, decahydronaphthalene, mineral oil, crude oils, etc.) which also includes aromatic hydrocarbon solvents (e.g., benzene, toluene, o-xylene, in-xylene, and p-xylene), halogenated hydrocarbon solvents (e.g., carbon tetrachloride, 1,2-dichloroethane, dichloromethane, chloroform, etc.), ester solvents (e.g., ethyl formate, methyl acetate, ethyl acetate, ethyl malonate, etc.), ketone solvents (e.g., acetone, methyl ethyl ketone, cyclohexanone, cyclopentanone, etc.), ether solvents (e.g., diethyl ether, dipropyl ether, diphenyl ether, tetrahydrofuran, 1,4-dioxane, etc.), amine solvents (e.g., propyl amine, diemylamine, triethylamine, aniline, pyridine), alcohol solvents (e.g., methanol, ethanol, 1-propanol, 1-butanol, 1-octanol, benzyl alcohol, trifluoroethanol), glycol solvents (glycerol, ethylene glycol, propylene glycol, m-cresol, etc.), acid solvents (e.g., acetic acid, hexanoic acid, etc.), carbon disulfide, nitrobenzene, N,N-dimethylformamide, N,N,-dimethylacetamide, dimethyl sulfoxide, N-methyl-2-pyrrolidone, acetonitrile, silicone solvents (e.g., silicone oils, polysiloxanes, cyclosilicones). A preferred solvent in the present compositions is water.

As used herein, “skin conditioning agent” refers to any material capable of protecting and treating compromised skin. Non-limiting examples of skin conditioning agents include one or more of beta-glucan, BP-Glucan PF, aluminium hydroxide gel, calamine, cocoa butter, cod liver oil, cyclopentasiloxane, dimethicone, dimethicone crosspolymer, dimethiconol, glycerin, kaolin, petrolatum, lanolin, mineral oil, shark liver oil, white petrolatum, talc, topical starch, zinc acetate, zinc carbonate, zinc oxide, live yeast cell derivatives, aldioxa, aluminum acetate, microporous cellulose, cholecalciferol, colloidal oatmeal, cysteine hydrochloride, dexpanthenol, protein hydrolysates, racemic methionine, sodium bicarbonate, vitamin A, buffered mixture of cation and anion exchange resins, corn starch, trolamine, bismuth subnitrate, boric acid, ferric chloride, polyvinylpyrrolidone-vinyl acetate, copolymers, sulfur, tannic acid, derivatives thereof and mixtures thereof.

As used herein, the term “salt” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecyl sulfonic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthalic acid, nicotinic acid, nitrous acid, oxalic acid, pelargonic acid, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

As used herein, the term “preservative” refers to any known biocompatible preservative that functions by inhibiting bacteria and/or fungi, and/or by inhibiting oxidation. Suitable preservatives can include, but are not limited to, antimicrobial agents and/or antioxidants. Suitable antimicrobial agents as preservatives can include but are not limited to benzoates, benzyl alcohol, sodium benzoate, n-alkyl dimethyl benzyl ammonium chloride, Caprylyl Glycol, Chlorphenesin, methylparaben, propylparaben, ethylhexylglycerin, phenoxyethanol, Phenoxyethanol and Caprylyl Glycol and Chlorphenesin, chlorocresol, potassium sorbate, sorbic acid, bronopol, methychloroisothiazolinone, methylisothiazolinone, sorbates, propionates, and nitrites. Suitable antioxidants can include, but are not limited to, vitamin C, butylated hydroxytoluene (BHT), sulphites, and vitamin E.

As used herein, “subject” or “individual” or “animal” or “patient” or “mammal,” refers to any subject, particularly a mammalian subject, for whom diagnosis, prognosis, or therapy is desired, for example, a human.

As used herein, “thickener” or “thickening agent” refers to any agent useful as an aid to thicken or add structure to a topical formulation. These agents impart physical stability and increased viscosity. Additionally, a thickener refers to one or more agents that, alone or in combination, result in a viscosity suitable for dermatologic applications. Thickening agents herein may be, for example, gums and natural polysaccharides, mineral thickeners, oils, and synthetic polymeric thickeners. Non limiting examples of thickeners suitable for use in the present topical compositions include, for example, one or more of Xanthan Gum, Cetyl Alcohol, PEG-100 Stearate, Glyceryl Stearate, and Magnesium Aluminum Silicate.

As used herein, a “treatment” of or “treating” a disease, disorder, or condition encompasses alleviation of at least one symptom thereof, a reduction in the severity thereof, or the delay or inhibition of the progression thereof. Treatment need not mean that the disease, disorder, or condition is totally cured. In this regard, treatment may include treating, preventing or ameliorating a disease, a symptom thereof or a condition related thereto. A useful composition herein needs only to reduce the severity of a disease, disorder, or condition, reduce the severity of symptoms associated therewith, provide improvement to a patient or subject's quality of life, or delay or inhibit the onset of a disease, disorder, or condition.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application. For example, “an” astringent component refers to both one astringent component or a mixture comprising two or more astringent components.

Unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained.

Throughout the application, descriptions of various embodiments use “comprising” language; however, it will be understood by one of skill in the art that in some specific instances, an embodiment can alternatively be described using the language “consisting essentially of” or “consisting of.”

Other terms as used herein are meant to be defined by their well-known meanings in the art.

### Biocompatible Topical Compositions

The presently described biocompatible topical compositions can be provided in any form, including, but not limited to, a gel, a cream, a lotion, an ointment, a foam, an aerosol, a powder, a solution, an emulsion, and a serum.

The compositions may comprise or consist of a combination of at least four bioactive elements such as, e.g., disodium acetyl glucosamine phosphate, such as lactobacillus/panax ginseng root extract ferment filtrate, dipeptide diaminobutyroyl benzylamide diacetate, hydrolyzed sericin, angelica polymorpha sinensis root extract, and beta-glucan, among others. The topical compositions may optionally comprise or consist of one or more additional agents. For example, the topical composition may optionally comprise or consist of a carrier, which may be a solvent, a chelating agent, a humectant, a surfactant, a viscosity adjusting agent, a glycosaminoglycan (GAG) stimulator, a buffering agent, a thickener, an emollient, an emulsifier, a skin conditioning agent, an anti-inflammatory agent, an anti-aging agent, an anti-wrinkle agent, a DNA repair agent, an antioxidant, a pH adjuster, a fragrance and/or a preservative. In addition, the topical composition may also comprise or consist of one or more biocompatible carrier, excipient, filler and/or diluent.

The presently provided biocompatible topical compositions may be free from a retinoid such as retinol.

The biocompatible topical compositions may also include known biocompatible carriers, excipients, fillers, and diluents that are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of all of which are hereby incorporated by reference in their entirety.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 18th Ed., Mack Publishing Co., Easton, Pa. (1990), both of which are incorporated by reference herein in their entirety.

Formulation 1, provided below in TABLE 1, is an exemplary topical composition according to the present subject matter:

TABLE 1

| Supplier | Trade Name | US INCI Name (PCPC) | Amount |
|---|---|---|---|
| Product Quest | DI Water | Water | 25.0400 |
| Univar USA Inc./OXEA | Butylene | Butylene Glycol | 1.0000 |
| Corporation | Glycol | | |
| Univar USA Inc./Brenntag | Propylene | Propylene Glycol | 8.0000 |
| | Glycol | | |
| Univar USA Inc./The Dow | Dipropylene | Dipropylene Glycol | 10.0000 |
| Chemical company | Glycol | | |
| Rita Coporation | Glycerin USP | Glycerin | 5.0000 |
| | 99.7% | | |
| Nexeo Solutions/Giles | Magnesium | Water | 0.5000 |
| Chemical | Sulfate | Magnesium Sulfate | |
| Morton Salt | Sodium | Sodium Chloride | 0.0100 |
| | Chloride USP | | |
| Univar USA Inc./Avantor | Sodium | Sodium Phosphate | 0.3000 |
| Performance Materials | Phosphate | | |
| | Monobasic | | |
| BASF Coporation/Cognis | Bronidox 1160 | Phenoxyethanol | 0.9000 |
| Induchem USA, Inc. | NovHyal | Glycerin | 0.1000 |
| | Biotech-(G) | Water | |
| | | Disodium Acetyl | |
| | | Glucosamine Phosphate | |
| Bioland | Bio-FRGE | Water | 0.2000 |
| | | Glycerin | |
| | | Lactobacillus/Panax | |
| | | Ginseng Root Extract | |
| | | Ferment Filtrate | |
| | | Caprylyl Glycol | |
| | | 1,2-Hexanediol | |
| Resources of Nature, LLC | DC Upregulex- | Water | 2.0000 |
| | Buddeja | Glycerin | |
| | Complex | Hydrolyzed Sericin | |
| | | Phospholipids | |
| | | Butylene Glycol | |
| | | Buddleja Davidii | |
| | | Meristem Cell Culture | |
| | | Xanthan Gum | |
| | | Phenoxyethanol | |
| | | Caprylyl Glycol | |
| | | Potassium Sorbate | |
| | | Hexylene Glycol | |
| DSM Nutrional Products, | Syn-Ake | Glycerin | 2.0000 |
| Inc. | | Water | |
| | | Dipeptide | |
| | | Diaminobutyroyl | |
| | | Benzylamide Diacetate | |
| Resources of Nature, LLC | DC Provesselin | Water | 1.0000 |
| | | Phospholipids | |
| | | Butylene Glycol | |
| | | Angelica Polymorpha | |
| | | Sinensis Root Extract | |
| | | Phenoxyethanol | |
| | | Caprylyl Glycol | |
| | | Potassium Sorbate | |
| | | Hexylene Glycol | |
| Botanicals Plus | BP-Glucan PF | Water | 2.0000 |
| | | Glycerin | |
| | | Beta-Glucan | |
| | | 1,2-Hexanediol | |
| | | Pentylene Glycol | |
| Induchem USA, Inc. | Unirepair T-43 | Water | 0.5000 |
| | | Butylene Glycol | |
| | | Acetyl Tyrosine | |
| | | Proline | |
| | | Hydrolyzed Vegetable | |
| | | Protein | |
| | | Adenosine Triphosphate | |
| | | PEG-35 Castor Oil | |
| | | PEG-12 Glyceryl Laurate | |
| | | PVP | |
| | | Disodium EDTA | |
| | | Tocopherol | |
| | | Acetaldehyde | |
| | | Glycol | |
| | | Diethylene Glycol | |
| | | 1,4-Dioxane | |
| Dow Corning Corporation | Dow Corning | Cyclopentasiloxane | 5.0000 |
| | 245 Fluid | | |
| Dow Corning Corporation | Dow Corning | Cyclopentasiloxane | 11.0000 |
| | 9011 Elastomer | PEG-12 Dimethicone | |
| | Blend | Crosspolymer | |
| | | Dimethylcyclosiloxanes | |
| | | Cyclotetrasiloxane | |
| Shin-Etsu Chemical Co., | KSG-210 | Dimethicone | 10.0000 |
| Ltd. | | Dimethicone/PEG-10/15 | |
| | | Crosspolymer | |
| | | Dipropylene Glycol | |
| | | Sodium Citrate | |
| | | Tocopherol | |
| Dow Corning Corporation | Dow Corning | Cyclopentasiloxane | 15.0000 |
| | 9546 Silicone | Dimethicone | |
| | Elastomer | Crosspolymer | |
| | Blend | Dimethicone/Vinyl | |
| | | Dimethicone | |
| | | Crosspolymer | |
| | | Dimethiconol | |
| | | Cyclotetrasiloxane | |
| PREMIER SPECIALTIES, | Lotus | Fragrance | 0.3500 |
| INC. | Fragrance-Low | Benzyl Alcohol | |
| | Allergen S14- | Hydroxycitronellal | |
| | 78513 | Citronellol | |
| | | Citral | |
| | | Limonene | |
| BASF Coporation | Retinol 50 C | Polysorbate 20 | 0.0000 |
| | | Retinol | |
| | | BHT | |
| | | BHA | |
| Induchem USA, Inc. | Uniprotect PT-3 | Panthenyl Triacetate | 0.1000 |
| | | Ethyl Linoleate | |
| | | Oleyl Alcohol | |
| | | Tocopherol | |
| | | Pantolactone | |
| | | Acetyl Aminopropanol | |
| | | Water | |
| | | Panthenyl Diacetate | |
| | | Panthenyl Acetate | |
| | | Ethanol | |
| | | Acetic Acid | |
| | | Total | 100.0000 |

Notably, the compositions according to the present subject matter may include more water than prior known formulations. In this regard, the compositions provided herein may include water in an amount of 20.0000% or more. The water may be present in an amount of from 20.0000% to 30.0000%, from 20.0000% to 27.5000%, from 20.0000% to 26.0000%, from 21.0000% to 30.0000%, from 21.0000% to 27.5000%, from 21.0000% to 26.0000%, from 22.0000% to 30.0000%, from 22.0000% to 27.5000%, from 22.0000% to 26.0000%, from 23.0000% to 30.0000%, from 23.0000% to 27.5000%, from 23.0000% to 26.0000%, 24.0000% to 30.0000%, from 24.0000% to 27.5000%, from 24.0000% to 26.0000%, or about 25.0000%.

In addition, the compositions may also include less than 0.5000% of disodium acetyl glucosamine phosphate, or a composition comprising the same. The disodium acetyl glucosamine phosphate or composition comprising the same may be present in any amount of from 0.0001% to 0.4900%, 0.0001% to 0.4000%, 0.00001% to 0.3000%, 0.0001% to 0.2000%, 0.0001% to 0.1000%, 0.1000%, 0.2000%, 0.3000%, 0.4000% or any other amount less than 0.5000%. According to the present subject matter, the disodium acetyl glucosamine phosphate may be present in a composition such as, for example, NOVHYAL® Biotech-(G).

In addition, a ginseng extract such as, for example, lactobacillus/panax ginseng root extract ferment filtrate, or a composition comprising the same may be present in any amount less than 0.7500%. For example, ginseng extract or composition comprising the same may be present in an amount of from 0.0001% to 0.7400%, 0.0001% to 0.7000%, 0.0001% to 0.6000%, 0.0001% to 0.5000%, 0.0001% to 0.4000%, 0.0001% to 0.3000%, 0.0001% to 0.2000%, 0.0001% to 0.1000%, 0.1000%, 0.2000%, 0.3000%, 0.4000%, 0.5000%, 0.6000%, 0.6500%, 0.7000%, or any other amount less than 0.7500%. According to the present subject matter the ginseng extract may be present in the form of lactobacillus/panax ginseng root extract ferment filtrate in a composition such as, for example, BIO-FRGE®.

The sericin may be present in the form of hydrolyzed sericin or a composition comprising sericin or hydrolyzed sericin. The sericin, hydrolyzed sericin or composition comprising the same may be present in any amount less than 2.0000%. For example, it may be present in an amount of from 0.0001% to 1.9999%, from 0.0001% to 1.8000%, from 0.0001% to 1.5000%, from 0.0001% to 1.2500%, from 0.0001% to 1.0000%, 0.1000%, 0.5000%, 1.0000%, 1.5000%, 1.7500%, 1.9000% or any other amount less than 2.0000%. According to the present subject matter, the sericin may be in a composition comprising sericin and stem cells such as, for example, DC Upregulex-Buddeja Complex.

When beta-glucan is utilized in a formulation according to the present subject matter, it may be present in any amount less than 3.0000%. For example, it may be present in an amount of from 0.0001% to 2.9999%, from 0.0001% to 2.8000%, from 0.0001% to 2.5000%, from 0.0001% to 2.2500%, from 0.0001% to 2.0000%, 0.1000%, 0.5000%, 1.0000%, 1.5000%, 1.7500%, 2.0000%, 2.2500%, 2.5000%, 2.7500%, or any other amount less than 3.0000%.

One or more of the DNA repair agents may be present in any amount less than 1.0000%. When more than one DNA repair agent is utilized, each may be in an amount of from 0.0001% to 0.9999%, from 0.0001% to 0.8000%, from 0.0001% to 0.7000%, from 0.0001% to 0.6000%, from 0.0001% to 0.5000%, 0.1000%, 0.2000%, 0.3000%, 0.4000%, 0.5000% or any other amount less than 1.0000%. According to the present subject matter, the one or more DNA repair agent may be Unirepair T-43 or Uniprotect PT-3.

### Methods of Treatment

Also provided herein are methods of using the biocompatible topical compositions herein for treating and preventing wrinkled, aged, uv-damaged or otherwise compromised skin.

The methods of treating, preventing, and ameliorating described herein may comprise or consist of topically administering one or more of the described biocompatible topical compositions to skin of a subject in need thereof.

Also provided is a method of treating, preventing or ameliorating a symptom associated with compromised skin that may comprise or consist of topically administering one or more of the described biocompatible topical compositions to skin of a subject in need thereof.

In addition, all of the methods described herein further comprise or consist of administering one or more additional compositions to the subject. The one or more additional compositions may be a skin cleanser; an oral composition for treating skin; a sunscreen such as, e.g., an organic sunscreen; a moisturizing composition; an exfoliating composition such as, e.g., an exfoliating polish; and/or a barrier protecting composition; or other composition suitable for addressing a symptom thereof or a condition related to compromised skin such as, e.g., a topical formulation comprising salicylic acid or benzoyl peroxide.

The methods may also further comprise or consist of administering or providing one or more additional therapies, including an at-home or office-administered concomitant device or therapies. Examples of at-home use devices include hand held ultrasound, devices for administering light (UV light therapies, red light therapies, laser therapies, and therapies utilizing intense non-coherent light sources, LLLT (low level laser or low level light therapy), LILT (low intensity light therapy), photobiostimulation, biostimulation (BIOS), photobiomodulation, photonic stimulation, photorejuvenation or the like) and/or electrical therapy based (electrophoresis, electrophoresis or the like). Non-limiting examples of office-administered therapies include any concomitant therapy administered under the supervision of a dermatologist or other professional skin care provider, such as administration of a topical composition utilizing a micro-needle technique.

Suitable micro-needle therapies include those using arrays of relatively small structures, sometimes referred to as micro-needles or micro-pins, for delivery and/or removal of therapeutic agents and other substances through the skin and other surfaces.

In addition, the methods described herein may also further include administering one or more homeopathic products to the subject. Homeopathic therapies are those in which very dilute form of an active substance is used to relieve similar symptoms in conditions resulting from different etiologies. Non-limiting examples of homeopathic products include over the counter compositions comprising or consisting of one or more of topical or oral forms of belladonna, nuxvomica, sulphur, arsenicum album, arsenicum bromatum, carbo animalis, causticum, hydrocotyle, kali bromatum, kali iodatum, kreosotum, lycopodium, nux vomica, petroleum, rhustoxicodendron, sepia, sulphur, echinacea, taraxacum, thuja occidentalis leafy twig, pine needle oil (pinus sylvestris) sambucus nigra flower calendula officinalis flower, abies balsamea leaf oil, echinacea angustifolia or the like.

### Dosage

The presently described biocompatible topical compositions can be topically administered in any form. A sufficient amount of the topical composition can be applied onto the affected area and surrounding skin, for example, in an amount sufficient to cover an affected area plus a margin of healthy skin or tissue surrounding the affected area, if possible, for example, a margin of about 0.5 inches. The compositions can be applied to any skin surface, including for example, facial skin, and the skin of the hands, neck, chest and/or scalp.

The compositions can be applied in a single, one-time application, once a week, biweekly, monthly, or from one to four times daily, for a period of time sufficient to alleviate symptoms, for example, for a period of time of one week, from 1 to 12 weeks or more, from 1 to 6 weeks, from 1 to 4 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day or twice per day. The presently described compositions can be topically administered once per day for a period of time from 1 week to 4 weeks, of from 1 week to 2 weeks, for 1 week, for 2 weeks, for 3 weeks, for 4 weeks, or for 4 weeks or more.

The presently described topical compositions can be applied in a therapeutically effective amount, for example an amount sufficient to cover an affected area plus a margin of healthy skin or tissue surrounding the affected area, for example, a margin of about 0.5 inches. Suitable amounts, for example, per application per affected area or cumulative daily dosage per affected area (for example two applications in a 24 hour period), can include, for example, from about 0.1 grams to about 6 grams; from about 0.2 grams to about 4.5 grams; from about 0.3 grams to about 4 grams; from about 0.4 grams to about 3.5 grams; from about 0.4 grams to about 3 grams; from about 0.4 grams to about 2.5 grams; from about 0.4 grams to about 2 grams; from about 0.4 grams to about 1.5 grams; from about 0.5 grams to about 1.5 grams; from about 0.5 grams to about 4.5 grams; 0.5 grams to about 4 grams; 0.5 grams to about 3.5 grams; 0.5 grams to about 3 grams; 0.5 grams to about 2.5 grams; 0.5 grams to about 2 grams; 0.5 grams to about 1 gram; about 0.5 grams; about 1 gram; about 1.5 grams; about 2 grams; about 2.5 grams; or about 3 grams.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

The compositions may be given in a single or multiple doses per time period, for example, daily, weekly, bi-weekly, or monthly. In an embodiment, the biocompatible compositions can be administered from one to four times per period, for example once daily or twice daily. In another embodiment, the present compositions may be administered once per week, for a period of from one to six weeks, for example for one week, for two weeks, for three weeks, for four weeks, five weeks, or for six weeks.

It is understood, however, that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

The optimal topical biocompatible formulations will be determined by one skilled in the art depending upon considerations such as the particular active agent combination and the desired dosage. See, e.g., “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the essential lipids.

The present biocompatible topical composition in accordance with the subject matter described herein may be a topical dosage form packaged in, for example, a multi-use or single-use package, including for example, a tube, a bottle, a pump, a container or bottle, a vial, ajar, a packet, or a blister package.

Single dosage kits and packages containing a once per day amount of the topical biocompatible composition may be prepared. Single dose, unit dose, and once-daily disposable containers of the present biocompatible compositions are contemplated as within the scope of the present subject matter.

The present topical biocompatible compositions remain stable in storage for periods including up to about 5 years, between about 3 months and about 5 years, between about 3 months and about 4 years, between about 3 months and about 3 years, and alternately any time period between about 6 months and about 3 years.

The presently described biocompatible topical composition in accordance with the subject matter described herein remains stable for up to at least 3 years at a temperature equal to 25° C. In an embodiment, the presently described topical formulation remains stable for at least 2 years at a temperature equal to 25° C. In an embodiment, the presently described biocompatible composition remains stable for at least 3 years at a temperature equal to 25° C. and at a humidity of up to 60% RH, for at least 2 years at a temperature equal to 25° C. and at a humidity of up to 60% RH, or for at least 18 months at a temperature equal to 30° C. and at a humidity of up to 75% RH. In a further embodiment, the presently described biocompatible composition in accordance with the subject matter described herein remains stable for an extended period of time when packaged in a multi-use container such as a bottle dispenser or the like, and exhibits equal to or even greater stability when packaged in a single-use package.

### EXAMPLE EMBODIMENTS

Embodiments disclosed herein include:

A. A topical composition, comprising at least four agents selected from the group consisting of a first glycosaminoglycan (GAG) stimulator, a second GAG stimulator, a first anti-aging agent, a second anti-aging agent, a cosmetic peptide, and a skin-conditioning agent; and optionally one or more additional agent, wherein the composition does not include a retinoid, and wherein the first GAG stimulator is not the same as the second GAG stimulator and the first anti-aging agent is not the same as the second anti-aging agent.
B. A kit comprising a topical composition according to A, including any of the additional elements 1-13 described below.
C. A method for improving skin hydration comprising topically administering a therapeutically effective amount of the topical composition according to A, including any of the additional elements 1-13 described below.

Embodiment A may have one or more of the following additional elements in any combination:

Element 1: A first GAG stimulator selected from the group consisting of disodium acetyl glucosamine phosphate (DAGP), hydrolyzed sericin, sericin, hyaluronic acid and derivatives or salts thereof, SUBLISKIN®, HYALUFIX®, STIMULHYAL®, SYN-GLYCAN®, KALPARIANE®, DC Upregulex, DC Upregulex-Buddeja Complex, glucosamine, N-acetyl glucosamine, Arctium lappa fruit extract, Eriobotrya japonica extract, Genkwanin, N-Methyl-L-serine, (−)-alpha-bisabolol or synthetic alpha-bisabolol, oat glucan, Echinacea purpurea extract, soy protein hydrolysate, Sinorhizobium Meliloti Ferment Filtrate, Calcium ketogluconate, Alpinia galanga leaf extract and tetradecyl aminobutyroylvalylaminobutyric urea trifluoroacetate.

Element 2: A second GAG stimulator is one or more GAG stimulator selected from the group consisting of disodium acetyl glucosamine phosphate (DAGP), hydrolyzed sericin, sericin, hyaluronic acid and derivatives or salts thereof, SUBLISKIN®, HYALUFIX®, STIMULHYAL®, SYN-GLYCAN®, KALPARIANE®, DC Upregulex, DC Upregulex-Buddeja Complex, glucosamine, N-acetyl glucosamine, Arctium lappa fruit extract, Eriobotrya japonica extract, Genkwanin, N-Methyl-L-serine, (−)-alpha-bisabolol or synthetic alpha-bisabolol, oat glucan, Echinacea purpurea extract, soy protein hydrolysate, Sinorhizobium Meliloti Ferment Filtrate, Calcium ketogluconate, Alpinia galanga leaf extract and tetradecyl aminobutyroylvalylaminobutyric urea trifluoroacetate.

Element 3: A first and/or second anti-aging agent selected from the group consisting of lactobacillus/panax ginseng root extract ferment filtrate, angelica polymorpha sinensis root extract, a flavonoid, CoQ10, inorganic sunscreens, vitamins and botanical extracts.

Element 4: A cosmetic peptide selected from the group consisting of argireline analogues, pentapetide leuphasyl, acetyl hexapeptide, pentapeptide-3V and dipeptide diaminobutyroyl benzylamide diacetate

Element 5: A skin-conditioning agent selected from the group consisting of beta-glucan, aluminium hydroxide gel, calamine, cocoa butter, cod liver oil, cyclopentasiloxane, dimethicone, dimethicone crosspolymer, dimethiconol, glycerine, kaolin, petrolatum, lanolin, mineral oil, shark liver oil, white petrolatum, talc, topical starch, zinc acetate, zinc carbonate, zinc oxide, live yeast cell derivatives, aldioxa, aluminum acetate, microporous cellulose, cholecalciferol, colloidal oatmeal, cysteine hydrochloride, dexpanthenol, protein hydrolysates, racemic methionine, sodium bicarbonate, vitamin A, buffered mixture of cation and anion exchange resins, corn starch, trolamine, bismuth subnitrate, boric acid, ferric chloride, polyvinylpyrrolidone-vinyl acetate, copolymers, sulfur, tannic acid, derivatives thereof and mixtures thereof.

Element 6: A one or more additional agent selected from the group consisting of a carrier, a solvent, a humectant, a surfactant, a viscosity adjusting agent, an additional glycosaminoglycan (GAG) stimulator, a buffering agent, a thickener, an emollient, an emulsifier, an additional skin conditioning agent, an anti-inflammatory agent, an additional anti-aging agent, an anti-wrinkle agent, a DNA repair agent, an antioxidant, a pH adjuster, a fragrance, a preservative, an excipient, a filler and a diluent.

By way of non-limiting example, exemplary combinations applicable to A include, but are not limited to, any of Elements 1-6 in combination with one or more of Elements 1-6.

### EXAMPLES

To facilitate a better understanding of the embodiments described herein, the following examples of various representative embodiments are given. In no way should the following examples be read to limit, or to define, the scope of the present disclosure.

### Example 1

TABLE 2, shown below, is a comparison of two formulations, one according to the present subject matter (Formulation 1, as above) and a second formulation that is outside the scope of the current subject matter (Formulation 2).

TABLE 2

| | | For- | For- |
|---|---|---|---|
| Supplier | Trade Name | mulation 2 | mulation 1 |
| Product Quest | DI Water | 19.89 | 25.04 |
| Univar USA Inc./ | Butylene Glycol | 1.0000 | 1.0000 |
| OXEA Corporation | | | |
| Univar USA | Propylene Glycol | 8.0000 | 8.0000 |
| Inc./Brenntag | | | |
| Univar USA Inc./The | Dipropylene Glycol | 10.0000 | 10.0000 |
| Dow Chemical company | | | |
| Rita Coporation | Glycerin USP 99.7% | 5.0000 | 5.0000 |
| Nexeo Solutions/Giles | Magnesium Sulfate | 0.5000 | 0.5000 |
| Chemical | | | |
| Morton Salt | Sodium Chloride USP | 0.0100 | 0.0100 |
| Univar USA | Sodium Phosphate | 0.3000 | 0.3000 |
| Inc./Avantor | Monobasic | | |
| Performance Materials | | | |
| BASF | Bronidox 1160 | 0.9000 | 0.9000 |
| Coporation/Cognis | | | |
| Induchem USA, Inc. | NovHyal Biotech- | 0.5000 | 0.1000 |
| | (G) | | |
| Bioland | Bio-FRGE | 0.7500 | 0.2000 |
| Resources of Nature, | DC Upregulex- | 3.0000 | 2.0000 |
| LLC | Buddeja Complex | | |
| DSM Nutrional | Syn-Ake | 2.0000 | 2.0000 |
| Products, Inc. | | | |
| Resources of Nature, | DC Provesselin | 2.0000 | 1.0000 |
| LLC | | | |
| Botanicals Plus | BP-Glucan PF | 3.0000 | 2.0000 |
| Induchem USA, Inc. | Unirepair T-43 | 1.0000 | 0.5000 |
| Dow Corning | Dow Corning 245 | 5.0000 | 5.0000 |
| Corporation | Fluid | | |
| Dow Corning | Dow Corning 9011 | 11.0000 | 11.0000 |
| Corporation | Elastomer Blend | | |
| Shin-Etsu Chemical Co., | KSG-210 | 10.0000 | 10.0000 |
| Ltd. | | | |
| Dow Corning | Dow Corning 9546 | 15.0000 | 15.0000 |
| Corporation | Silicone Elastomer | | |
| | Blend | | |
| PREMIER | Lotus Fragrance-Low | 0.3500 | 0.3500 |
| SPECIALTIES, INC. | Allergen S14-78513 | | |
| BASF Coporation | Retinol 50 C | 0.3000 | 0.0000 |
| Induchem USA, Inc. | Uniprotect PT-3 | 0.5000 | 0.1000 |
| | Total | 100.0000 | 100.0000 |

Formulation 1 is superior to formulation 2. Evidence of the superior properties of formulation 1 can be found in the below examples.

### Example 2

The effect of topical formulation 1 on biomechanical properties of skin was compared to the effect of topical formulation 2 in a monadic, seven-day proof of concept study.

Seventeen female subjects (discontinuance=0), age 40-65 years, Fitzpatrick Skin Types I-VI in approximately equal distribution, applied serums to each volar forearm in the course of the study. Subject statistics are reproduced in TABLE 3.

TABLE 3

| Variable | n | Mean ± SD | Min | Max |
|---|---|---|---|---|
| Age (years) | 17 | 52.17 ± 5.94 | 42 | 64 |
| Height (in) | 17 | 64.29 ± 2.46 | 60 | 68 |
| Weight (lbs) | 17 | 174.70 ± 32.12 | 120 | 240 |
| Ethnicity | 17 | Hispanic or Latino | 1 | 5.9% |
| | | Not Hispanic or Latino | 16 | 94.1% |
| Race | 17 | Asian | 2 | 11.8% |
| | | Black or African American | 6 | 35.3% |
| | | White | 9 | 52.9% |
| Fitzpatrick Skin | 17 | Skin Type II | 3 | 17.6% |
| Type | | Skin Type III | 5 | 29.4% |
| | | Skin Type IV | 3 | 17.6% |
| | | Skin Type V | 5 | 29.4% |
| | | Skin Type VI | 1 | 5.9% |
| Body Skin Type | 17 | Dry | 4 | 23.5% |
| | | Normal | 13 | 76.5% |

Before commencement of the seven-day proof of concept study, a seven day screen/washout procedure was observed. Support products used for the duration of the study and for the washout period were a gentle cleanser and a sunscreen applied every morning.

After the washout period, subjects were assigned formulation 1 or formulation 2 per a prepared randomization code. Serums were applied once in-clinic at baseline visits, and then morning and evening daily to the assigned area of volar forearms. Corneometer readings were taken immediately before first application to establish baseline skin hydration (“Baseline”). Corneometer readings were also taken immediately after application (“Immediate”), three days later (“Day 3”) and seven days later (“Day 7”).

During the study, subjects avoided prolonged sun exposure events and did not participate in any other clinical studies. Subjects did not have any acute or chronic diseases or medical conditions, including dermatological problems, which would potentially compromise study outcomes, including but not limited to cancer, AIDS, insulin dependent diabetes, renal impairment, mental illness, drug addition, alcohol addiction; had no history of allergic reactions, skin sensitization and/or known allergies to cosmetic ingredients, toiletries, sunscreens, etc.; were not immunocompromised; had not started hormone replacement theory within three months of the preceding screening visit; were not pregnant, lactating, or planning to become pregnant; and had not modified their contraceptive method during the course of the study or within the previous three months.

Results of the corneometer readings are reproduced in TABLE 4.

TABLE 4

| | | Formulation 2 | | | Formulation 1 | |
|---|---|---|---|---|---|---|
| | | | Mean % | | | Mean % |
| | | | Improvement | | | Improvement |
| Time Point | n | Mean ± SD | over Baseline | n | Mean ± SD | over Baseline |
| Baseline | 17 | 38.27 ± 7.07 | — | 17 | 36.68 ± 7.50 | — |
| Immediate | 17 | 61.45 ± 10.42 | 63.92% | 17 | 62.41 ± 13.00 | 74.41% |
| Day 3 | 17 | 51.56 ± 9.18 | 36.98% | 17 | 50.77 ± 9.22 | 42.65% |
| Day 7 | 17 | 42.77 ± 7.43 | 13.21% | 17 | 45.42 ± 9.25 | 26.51% |

Difference comparison of the results of the corneometer readings instrumental evaluation are reproduced in TABLE 5.

TABLE 5

| | | Formulation 2 | | Formulation 1 | |
|---|---|---|---|---|---|
| | | Mean | | Mean | |
| | | Difference ± SD | | Difference ± SD | P_{T}-Value |
| Time Point | n | from baseline | n | from baseline | 2 vs 1 |
| Immediate | 17 | 23.18 ± 10.35 | 17 | 25.73 ± 12.69 | 0.526 |
| Day 3 | 17 | 13.29 ± 8.90 | 17 | 14.09 ± 10.38 | 0.811 |
| Day 7 | 17 | 4.50 ± 6.18 | 17 | 8.74 ± 9.05 | 0.122* |

*Indicates a confidence interval of 85% or higher.

When hydration assessments using Corneometer were compared, a confidence level of 85% or higher was observed at Day 7 in the hydration improvements, with Formulation 1 of the present disclosure outperforming Formulation 2. Formulation 1 shows superior hydration properties at Day 7.

Under the conditions of the experiment, both formulations showed the potential to improve skin hydration immediately and over time. At Day 7, Formulation 1 outperformed Formulation 2 at an 85% confidence level.

All publications cited in the specification are indicative of the level of skill of those skilled in the art to which the presently described subject matter pertains. All of these publications are hereby incorporated by reference herein to the same extent as if each individual publication were specifically and individually indicated as being incorporated by reference.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

1. A topical composition comprising:
disodium acetyl glucosamine phosphate (DAGP), sericin (hydrolyzed or non-hydrolyzed), lactobacillus/panax ginseng root extract ferment filtrate, dipeptide diaminobutyroyl benzylamide diacetate as a cosmetic peptide, and a skin-conditioning agent; and
optionally one or more additional agent, and
wherein the composition does not include a retinoid.

2. The topical composition according to claim 1, wherein the first GAG stimulator is one or more GAG stimulator selected from the group consisting of disodium acetyl glucosamine phosphate (DAGP), hydrolyzed sericin, sericin, hyaluronic acid and derivatives or salts thereof, SUBLISKIN®, HYALUFIX®, STIMULHYAL®, SYN-GLYCAN®, KALPARIANE®, DC Upregulex, DC Upregulex-Buddeja Complex, glucosamine, N-acetyl glucosamine, Arctium lappa fruit extract, Eriobotrya japonica extract, Genkwanin, N-Methyl-L-serine, (−)-alpha-bisabolol or synthetic alpha-bisabolol, oat glucan, Echinacea purpurea extract, soy protein hydrolysate, Sinorhizobium Meliloti Ferment Filtrate, Calcium ketogluconate, Alpinia galanga leaf extract and tetradecyl aminobutyroylvalylaminobutyric urea trifluoroacetate.

3. The topical composition according to claim 1, wherein the second GAG stimulator is one or more GAG stimulator selected from the group consisting of disodium acetyl glucosamine phosphate (DAGP), hydrolyzed sericin, sericin, hyaluronic acid and derivatives or salts thereof, SUBLISKIN®, HYALUFIX®, STIMULHYAL®, SYN-GLYCAN®, KALPARIANE®, DC Upregulex, DC Upregulex-Buddeja Complex, glucosamine, N-acetyl glucosamine, Arctium lappa fruit extract, Eriobotrya japonica extract, Genkwanin, N-Methyl-L-serine, (−)-alpha-bisabolol or synthetic alpha-bisabolol, oat glucan, Echinacea purpurea extract, soy protein hydrolysate, Sinorhizobium Meliloti Ferment Filtrate, Calcium ketogluconate, Alpinia galanga leaf extract and tetradecyl aminobutyroylvalylaminobutyric urea trifluoroacetate.

4. The topical composition according to claim 1, wherein the first and/or second anti-aging agents is one or more anti-aging agent selected from the group consisting of lactobacillus/panax ginseng root extract ferment filtrate, angelica polymorpha sinensis root extract, a flavonoid, Coenzyme Q10, inorganic sunscreens, vitamins and botanical extracts.

5. The topical composition according to claim 1, wherein the cosmetic peptide is one or more cosmetic peptide selected from the group consisting of argireline analogues, pentapeptide leuphasyl, acetyl hexapeptide, pentapeptide-3V and dipeptide diaminobutyroyl benzylamide diacetate.

6. The topical composition according to claim 1, wherein the skin-conditioning agent is selected from the group consisting of Angelica Polymorpha Sinesis Root Extract, beta-glucan, aluminium hydroxide gel, calamine, cocoa butter, cod liver oil, cyclopentasiloxane, dimethicone, dimethicone crosspolymer, dimethiconol, glycerine, kaolin, petrolatum, lanolin, mineral oil, shark liver oil, white petrolatum, talc, topical starch, zinc acetate, zinc carbonate, zinc oxide, live yeast cell derivatives, aldioxa, aluminum acetate, microporous cellulose, cholecalciferol, colloidal oatmeal, cysteine hydrochloride, dexpanthenol, protein hydrolysates, racemic methionine, sodium bicarbonate, vitamin A, buffered mixture of cation and anion exchange resins, corn starch, trolamine, bismuth subnitrate, boric acid, ferric chloride, polyvinylpyrrolidone-vinyl acetate, copolymers, sulfur, tannic acid, derivatives thereof and mixtures thereof.

7. The topical composition according to claim 1, wherein the one or more additional agent is selected from the group consisting of a carrier, a solvent, a humectant, a surfactant, a viscosity adjusting agent, an additional glycosaminoglycan (GAG) stimulator, a buffering agent, a thickener, an emollient, an emulsifier, an additional skin conditioning agent, an anti-inflammatory agent, an additional anti-aging agent, an anti-wrinkle agent, a DNA repair agent, an antioxidant, a pH adjuster, a fragrance, a preservative, an excipient, a filler and a diluent.

8. A kit, comprising the topical composition according to claim 1.

9. A method for improving skin hydration comprising:
topically administering a therapeutically effective amount of the topical composition according to claim 1 to skin of a subject in need thereof.

10. The topical composition according to claim 1, wherein the sericin (hydrolyzed or non-hydrolyzed) is in complex with buddleja davidii meristem cell culture.

11. The topical composition according to claim 1, further comprising a plant derived growth factor derived from fermented Korean red ginseng.
